(19) United States
(12) Patent Application Publication
(10) Pub. No.: US 2023/0140497 A1
(43) Pub. Date: May 4, 2023

(54) NUTRITIONAL COMPOSITION FOR RENAL SUPPORT

(71) Applicant: Kate Farms, Inc., Santa Barbara, CA (US)

(72) Inventors: Melissa Bailey, Philadelphia, PA (US); Vanessa Millovich, Laguna Niguel, CA (US); Susan Ray, Temecula, CA (US); Zhaozhi Huang, Goleta, CA (US); Erin Lamb, Santa Barbara, CA (US)

(21) Appl. No.: 17/453,053

(22) Filed: Nov. 1, 2021

(51) Int. Cl. A23L 33/00; A23L 33/16; A23L 33/185; A23L 33/21; A23L 33/15; A23L 33/125

(52) U.S. Cl. CPC A23L 33/40; A23L 33/16; A23L 33/185; A23L 33/21; A23L 33/15; A23L 33/125

(57) ABSTRACT

A nutritional composition for enteral and oral feeding provides sole-source or supplemental nutrition via a specialized formula for people on dialysis due to chronic kidney disease (CKD) or acute kidney injury (AKI), the formula being a calorically dense, high protein formula with optimized electrolyte content for people on dialysis due to CKD or AKI, while being entirely plant-based and suitable for vegan, lactose-free, dairy-free, and gluten-free diets. The nutritional composition comprises pea protein, a balanced fat blend (e.g., sunflower, flaxseed, and medium chain triglycerides), an easy to digest carbohydrate blend (e.g., agave syrup, brown rice syrup solids), with agave inulin fiber, and micronutrients. The nutritional composition provides relatively high amounts (e.g., compared to standard formulas) of certain micronutrients, including vitamin B6, vitamin B12, zinc, and/or selenium, and relatively low amounts of sodium, potassium, and phosphorus to meet the needs of CKD patients on dialysis.

### BACKGROUND OF THE INVENTION

Chronic Kidney Disease (CKD) is characterized by gradual loss of kidney function over time. It is estimated that 15% of adults in the U.S. have CKD, with approximately 750,000 suffering from end stage renal disease (ESRD). Severity ranges from Stage 1, mild kidney damage, to Stage 5, ESRD requiring dialysis or transplant. The two main risk factors for development of CKD are Type II Diabetes and Hypertension.

Acute Kidney Injury (AKI) is characterized by sudden decline of kidney function. An AKI can range from mild to severe, and sometimes require dialysis or continuous renal replacement therapy (CRRT). It is estimated that two-thirds of patients in the intensive care unit (ICU) develop AKI as part of multi-organ dysfunction syndrome. AKI is also known as Acute Renal Failure.

At the same time, liquid nutrition supplements are well-known and have been readily available in stores and otherwise distributed for medical purposes for many years. These compositions come in various flavors and can be consumed in addition to a regular diet, as a replacement for meals in a day, or in lieu of other food altogether. Initially, they were developed mostly for use with medical supervision by the elderly, patients recovering from illness, and others who need help putting on or retaining weight or otherwise meeting their nutritional requirements, but more have recently been marketed for use by healthy adults.

In the medical field, these compositions are used to treat patients with a variety of ailments that have difficulty digesting foods and have decreased nutrient intake.

In addition, food allergies are increasingly common. Common allergens consist of milk (including whey, casein, and lactose), wheat, soybeans, eggs, peanuts, tree nuts, fish, shellfish, and corn. As nutrients are necessary for proper growth and development, removing them from a regular diet leads to malnutrition issues. In particular, removal of milks and/or milk proteins, eggs, and soybeans from diet of children can often have adverse consequences. Currently, products in the markets utilize corn sweeteners, corn starches, synthetic corn derivatives and corn oils, many of which are produced from genetically modified sources. In addition, these products utilize the hydrolyzed protein of milk, whey or casein making their products unavailable to those patients with galactosemia, who are lactose intolerant or have a milk allergy.

### SUMMARY OF THE INVENTION

In the light of above-mentioned problems, there is a need for a product that addresses the issues faced by people and patients who want to rely upon a plant-based, vegan formula, in which the formula is free of common allergens and synthetic ingredients. Further, there is a need for a plant-based composition comprising plant proteins, phytonutrients, beneficial fats, quality carbohydrate sources and fibers, and are suitable for both oral and enteral administration.

Additionally, there is a need for a plant-based nutrition formula that is specifically intended for patients with CKD or AKI.

There is research that suggests that plant proteins, and in some instances pea protein specifically, improve the health outcomes for patients with CKD and AKI. Generally, plant-based proteins have a role in kidney health and are thought to support kidney function.

Plant-based proteins have a low net endogenous acid load which is thought to mitigate metabolic acidosis in CKD patients and potentially slow the progression of kidney disease. The plant proteins have a higher proportion of basic amino acids relative to the sulfur containing amino acids that present a higher acid load. Benefits of a lower acid load and reduced metabolic acidosis support bone health in patients with kidney disease. Plant phosphorus is bound to phytate and is less bioavailable than animal phosphorous; consequently, many plant-based protein foods have a favorable protein to phosphorous ratio.

Patients with CKD are often placed on a renal diet, which may be used to restrict protein (in certain situations), sodium, potassium, and phosphorus. Recent findings indicate that while these restrictions are considered crucial to successful health outcomes of the patient, a plant-based diet may offer benefits to the patient versus a renal diet alone. Renal diets are used to manage CKD specifically but have not been shown to be effective in preventing comorbidities that commonly occur and include cardiovascular disease. Patients on a renal diet often restrict foods such as fruits and vegetables that contain phytonutrients (plant-based antioxidant compounds), since these contain micronutrients and/or other naturally occurring compounds at levels that are limited in this type of diet. Instead, the individual on the renal diet may choose more highly processed food items. Dietary components or attributes are thought to have a role in positive health outcomes for the renal patient. These components are contained in a plant-based diet and carefully designed for the renal patient. This approach includes plant-based protein, fiber, carbohydrate, fats, phytonutrients, as well as the required micronutrients-vitamins and minerals.

Recommendations for dietary protein intake levels and amounts are based on the stage of CKD that a patient is in and whether the patient is on dialysis. In CKD stages 1-4, lower protein is recommended. On the other hand, once on dialysis, a higher protein formula could be recommended. For those using an oral nutritional supplement (ONS) or medical food via feeding tube to support their nutritional needs, whey and/or casein protein have been the primary sources of protein available or offered. Formulas containing animal-based proteins may contain or be devoid of cholesterol and/or saturated fat depending on design. A review of randomized controlled trials suggested that replacing animal protein with plant protein, for 1-2 servings per day, decreased cholesterol levels. For management of cardiovascular disease, a formula devoid of cholesterol and saturated fat supports a healthy plasma cholesterol level and is beneficial. For the CKD patient, consumption of a plant-based protein diet that is well balanced and complies with the dietary renal guidelines can support reduced risk of disease progression.

Composition of the gut microbiome plays a significant role in the development, progression, and management of chronic disease. High fiber intake, specifically soluble, fermentable fiber, may lead to the increased production of short chain fatty acids (SCFA) and bile excretion. This may also help to decrease serum cholesterol and improve insulin sensitivity. An increase in fiber intake can shift the microflora for the positive, which may help trap and excrete nitrogen, thus reducing urea in the bloodstream. Fiber reduces the fermentation time of protein in the intestinal tract which results in decreased ammonia, phenols, and indoles that lead to uremic toxicity and inflammation.

Omega-3 fatty acids are known to reduce inflammation, serum triglycerides, blood pressure and heart rate while increasing serum HDL cholesterol. Plant fats have anti-atherogenic properties and offer benefits for renal health. Plant-derived omega-3 fatty acids, known as alpha-linoleic (α-linoleic) acids, are commonly found in vegetable oils, as well as in foods such as flaxseeds and chia seeds. Plant-based omega-3 fatty acids, such as flaxseed, may be beneficial for individuals who progress to ESRD and require dialysis. There is a benefit to consuming plant-based omega-3 fatty acids to reduce CVD, comorbid disease and all-cause mortality in CKD and/or ESRD patients.

Plants contain many phytonutrients, which function as antioxidants. In CKD patients, antioxidants have shown to reduce oxidative stress and possibly improve kidney function. While more clinical data is needed, the overall health benefit of phytonutrients is positive.

Based on this understanding, the present invention relates to nutritional compositions for use in both enteral and oral feeding that are allergen-free, plant-based and preferably entirely plant-based and vegan, organic, free of ingredients from genetically modified organisms (GMOs), and free of synthetic ingredients. These nutritional compositions should comprise a protein source (e.g., pea protein or hydrolyzed pea protein), phytochemical extracts, fatty acids, organic ingredients free of common allergens and carbohydrates and other ingredients sourced from corn, and prebiotic fibers. In addition, the compositions should avoid any “junk” ingredients that have been previously used to inexpensively meet macro or micronutrient levels (or other ingredients for flavor, color, viscosity, etc). In addition, the nutritional composition is preferably calorically dense so that nutrition needs can be met with less volume to support those with volume restrictions or intolerances. This is especially helpful for individuals with some renal system impairment such as CKD. In one example, the caloric density is approximately 1.8 kcal/mL

The presently disclosed nutritional composition for enteral and oral feeding provides sole-source or supplemental nutrition via a specialized formula for use in patients with CKD or AKI in addition to being entirely plant-based and suitable for plant-based, vegan, lactose-free, dairy-free, and gluten-free diets. The nutritional composition has a low glycemic index while excluding artificial sweeteners, flavors, corn syrup, or sugar alcohols and instead using natural organic vanilla flavor and agave syrup. Moreover, the nutritional composition provides the benefits of a plant-based diet but does not contain any lactose or milk protein, improving ease of digestion. The composition includes plant-based omega-3 for cardiovascular health and phytonutrients (having antioxidant properties) in a clinically effective amount for immune support. Naturally occurring arginine is also included for nitric oxide generation and many other essential benefits including toxin elimination, immune system maintenance, and to support nutritional needs of individuals with wounds for faster wound healing, to name a few examples. The composition does not contain any of the common allergens. Prebiotic soluble fiber is included to support the gut microbiome.

More particularly, the presently disclosed nutritional composition is a nutritionally complete enteral medical food formula made with organic ingredients and phytonutrient extracts and concentrates, including pea protein (e.g., organic yellow pea), a balanced fat blend (e.g., sunflower, flaxseed, and medium chain triglycerides derived from coconut), an easy to digest carbohydrate blend of complex and simple carbohydrates (e.g., agave syrup, brown rice syrup solids, pea starch), with agave inulin fiber, and micronutrients specifically formulated to support the nutrition needs of patients with CKD or AKI. The formula is plant-based, vegan, free of major allergens (milk, wheat, soy, eggs, peanuts, tree nuts, fish, shellfish), and free of corn-derived carbohydrates, non-GMO, kosher, and made with a blend of phytonutrients. The composition also has a pleasing taste and can be taken orally. The nutritional composition meets a need within the medical nutrition therapy market as the only plant-based vegan formula designed for use in patients with CKD or AKI. Previously, there were no sole-source nutrition, renal-specific formulas that contained entirely plant-based ingredients, and the nutritional composition is the first entirely plant-based, sole-source nutrition formula intended for patients on dialysis due to CKD or AKI and the first that does not use artificial sweeteners and sugar alcohols. The nutritional composition meets expert recommendations for nutrition support in CKD or AKI patients, including those undergoing dialysis. The nutritional composition is a calorically dense, high protein formula with optimized electrolyte content for people on dialysis due to CKD or AKI.

Patients with CKD on dialysis are at higher risk of deficiency of certain micronutrients, including water soluble vitamins, due to the micronutrients' removal during dialysis. To meet the needs of CKD patients on dialysis, the nutritional composition is designed with increased amounts of these micronutrients (e.g., vitamin B6, vitamin B12, zinc, selenium) per 250 mL serving (e.g., compared to standard formulas not designed for use in patients with CKD or AKI).

Moreover, the nutritional composition has optimized electrolyte content for people on dialysis due to CKD or AKI, for example, comprising lower amounts of sodium, potassium, and phosphorus (e.g., compared to standard formulas not designed for use in patients with CKD or AKI) to help manage imbalances faced by patients with CKD or AKI. These lower levels of sodium, potassium, and phosphorus further decrease renal stress. In one example, the nutritional composition comprises 1000-1400 mg of potassium per 1 L of the composition, 1000-1400 mg of sodium per 1 L of composition, 760-800 mg of phosphorous per 1 L of the composition, and/or 880-1000 mg of chloride per 1 L of the composition.

The nutritional composition is designed to promote glycemic control. It is estimated that up to 30% of patients with Type 1 Diabetes and up to 40% of patients with Type 2 Diabetes will develop kidney failure. Low glycemic index diets have been demonstrated to lead to statistically significant improvements in A1c and fasting glucose compared to a high glycemic index diet. In a randomized, crossover trial of overweight or obese adults with CKD, a low glycemic index (<45) with low carbohydrate diet (<40% kcal from carbohydrate) was shown to increase glomerular filtration rates. The nutritional composition has a low glycemic index. The composition contains a blend of complex and simple carbohydrates to create a favorable blood glucose response, including organic agave syrup, which is fructose-based and helps contribute to the composition's low glycemic index. In one example, the nutritional composition has a glycemic index of 42+/−5.

CKD is associated with a dysbiotic gut microbiota characterized by reduced diversity and an imbalance with a decrease in commensal bacteria and an increase in pathobionts and uremic toxin-producing bacteria. Restoration of a healthy gut microbiota in CKD and uremic patients is relevant in the area of nephrology. Modifiers of gut microbiota in uremic patients depend on the duration of the diet and nutritional composition. Plant-based diets and more specifically ingredients offered in a plant-based diets are key to supporting a healthy microbiome and to manage the changes that occur with progressing renal disease.

The nutritional composition preferably comprises a prebiotic fiber (e.g., agave inulin), which is easy to digest, promotes the growth of beneficial bacteria in the gut such as bifidobacteria, and reduces inflammation. Agave inulin is a soluble, prebiotic source of long chain fermentable fiber. In a study of healthy adults, agave inulin intakes of 5.0 g and 7.5 g per day were shown to positively alter the gut microbiome. In addition, formulas with prebiotic soluble fiber appear to be more effective in reducing diarrhea versus formulas that contain a blend of soluble and insoluble fiber, and high fiber diets have been associated with improvements in glycemic control parameters (fasting glucose and A1c) compared to low fiber diets. In one embodiment, the composition includes 1616 g per 1 L of composition of prebiotic soluble fiber from organic agave inulin.

The nutritional composition also uses high quality, plant-based protein such as pea protein. Many medical conditions require higher protein intakes (e.g., infection, liver and kidney disease during dialysis). After surgery, patients have elevated metabolic rates requiring both energy and protein for tissue repair. Severe acute illness, burn injury, end-stage renal disease during dialysis, and nursing home residents all require higher proteins needs. Patients with CKD on dialysis require higher protein intakes to support lean body mass, to make up for protein losses (albumin) during treatment and to make up for muscle loss, or enhanced tissue catabolism caused by inflammatory cytokines or elevated basal metabolic rates. This can be met by providing a formula with a higher caloric density and with more calories coming from dietary protein.

Thus, in one example, the nutritional composition has a caloric density of 1.8 kcal/mL, and 18% of its calories provided by high-quality protein such as pea protein. In fact, 100% of the protein in the nutritional composition can be from organic yellow pea.

Pea protein isolate is unique (among plant proteins) in having a high concentration of lysine, branched chain amino acids, arginine, and glutamine/glutamic acid. The percent content of amino acids in pea protein isolate is provided below, in Table 1.

TABLE 1
Percent Content of Amino Acids in Pea Protein Isolate

| Amino Acid | Benefit | % |
|---|---|---|
| Lysine | Human growth; Bone health | 7.2% |
| Branched-chain amino acids | Maintenance of muscle protein | 17.9% |
| Arginine | Physical effort; Immune | 8.7% |
| | system efficiency | |
| Glutamine + Glutamic acid | Source of energy for | 16.8% |
| | muscles during stress | |

Branched-chain amino acids are essential nutrients that the body obtains from proteins found in food, especially meat, dairy products, and legumes. They include leucine, isoleucine, and valine. “Branched-chain” refers to the chemical structure of these amino acids. Branched-chain amino acids have been used for medicine, as they stimulate the building of protein in muscle and possibly reduce muscle breakdown and also seem to prevent faulty message transmission in the brain cells of people with advanced liver disease, mania, tardive dyskinesia, and other types of disease.

Arginine is a semi-essential amino acid that is vitally important for nitric oxide generation and many other essential benefits including toxin elimination, immune system maintenance and faster wound healing. Pea protein is unique in that it contains almost three times the amount of arginine versus milk proteins like whey. High arginine plasma levels are critical for growth and development, and several studies of muscle growth in weightlifters using whey protein versus pea protein showed identical results in the anabolic effects on muscle tissue.

Moreover, the nutritional composition is designed to support patients with CKD or AKI while enhancing tolerability and digestibility. Due to its unique globular and low solubility characteristics, pea protein is considered an “intermediate” protein in digestibility, somewhere between whey (fast) and casein (slow). Pea protein also provides a high ease of digestion, with several studies showing that the true digestibility of pea protein (over 95%) is very similar to casein and eggs. Casein forms a “curd” in the stomach due to low stomach pH. Pea protein forms a similar but less strong curd that allows easier and quicker digestion and passage into the small intestine.

The organic, plant-based protein used in the nutritional composition has a Protein Digestibility Corrected Amino Acid Score (PDCAAS) of 1.0.

The need for higher protein intakes in CKD and AKI patients has historically been addressed by either whey and/or casein protein. At the same time, plant-based, vegan, and/or an allergy/intolerance to dairy protein has driven the use of formulas containing soy protein or a blend of plant-based proteins (not from soy). However, these formulas have a lower PDCAAS (e.g., below 1.0) and require decanting to remove phosphorus and/or potassium in order to meet the dietary needs of the patient with CKD or AKI.

In one example, the primary protein source is from organic yellow pea with additional amino acids to provide a complete amino acid profile, comparable to milk and soy, without the inherent allergenicity.

Preferably, the nutritional composition provides 80 g/L of plant-based protein, with a complete amino acid profile and a PDCAAS of 1.0, and arginine at 7.2 g/L.

The nutritional composition contains no non-nutritive sweeteners, sugar alcohols, or aspartame.

The nutritional composition is also free of the most common food allergens (e.g., milk, wheat, soy, eggs, peanuts, tree nuts, fish, and shellfish) and corn-derived carbohydrates and is gluten-free and suitable for patients with gluten allergies or Celiac disease. Ninety million Americans follow a gluten-free diet, and 24 million people need to avoid gluten for medical reasons, according to the Center for Celiac Disease Research and Treatment.

CKD patients have low-grade inflammation and oxidative stress associated with progression of renal dysfunction and complications of CKD such as atherosclerosis, cardiovascular disease risk and/or protein-energy wasting. The dysbiotic gut microbiome in CKD favors pathobionts overgrowth, together with an associated increase in intestinal barrier permeability that can contribute to systemic inflammation and oxidative stress in CKD patients through translocation of bacteria and bacterial products into the systemic circulation. The type of diet key to support reduced inflammation includes consumption of fruits and vegetables with naturally occurring phytonutrients as well as vitamins and other key antioxidants. Intake of these nutrients is associated with lower levels of inflammatory markers. Western diets rich in animal proteins and animal fats stimulate overgrowth of proteolytic bacteria, which results in dysbiosis and the accumulation of proteolytic-derived uremic toxins that promote CKD progression. Inclusion of plant-based phytonutrients from clean sources of plant based fruits, vegetables, herbs provide healthy gut microbiome support for the CKD patient.

Moreover, the composition comprises a phytonutrient blend from organic sources, including phytochemical extracts and concentrates, which are high in antioxidant properties and help maintain quality and flavor and reduce oxidation and ingredient degradation throughout the shelf life of the composition. A 100-150 milligram (mg) dose of one phytonutrient blend was shown to reduce markers of oxidative stress in healthy adult subjects.

The nutritional composition preferably comprises a blend of essential Omega-6 (e.g., sourced from high linoleic sunflower oil and/or flaxseed oil) and Omega-3 fatty acids (e.g., α-linolenic acid (ALA) sourced from flaxseed oil), plus the easily digestible organic medium chain triglycerides (MCTs) (for example, derived from organic coconut oil). Omega-6 is an essential fatty acid. Omega-3 has many anti-inflammatory properties. An omega 6:3 ratio of less than 4:1 has been shown to be beneficial for reducing mortality and decreasing inflammation. MCTs are easily digested and provide an immediate energy source via a portal vein, MCT oil helps to facilitate fat absorption, and the highly refined organic MCT source does not cause stomach upset upon digestion, unlike many synthetic versions. Additionally, flaxseed oil, MCT oil, and sunflower oil provide viscosity and improve the mouthfeel of the composition. Preferably, the nutritional composition has an omega 6:3 ratio ranging from 3:1 to 4:1.

In one embodiment, the nutritional composition comprises pea starch, which increases viscosity and improves stability, helping to keep the composition in solution to flow smoothly through a feeding pump.

The nutritional composition might also comprise locust bean gum, which is a stabilizer that increases viscosity, improves stability, and improves mouthfeel.

The nutritional composition might further comprise rosemary extract, which is an antioxidant that helps to maintain the quality and flavor of the composition and reduce oxidation and ingredient degradation throughout the composition's shelf life.

In addition, brown rice syrup solids can be added. These are carbohydrates that, at high dextrose equivalents (DE), prevent viscosity of the composition from becoming too high and maintain a relatively low viscosity. For example, the nutritional composition might have brown rice syrup solids with a DE of 20-30.

Sunflower lecithin can be added. This is an emulsifier that helps keep the composition in solution to flow smoothly through a feeding pump.

Patients who require enteral nutrition therapy often seek out products that are GMO-free. The nutritional composition uses only non-GMO based ingredients unlike many other enteral products.

The nutritional composition also uses organic (e.g., Certified Organic by Quality Assurance International (QAI)). Generally, organic ingredients that are free from pesticides, artificial fertilizers, antibiotics, or hormones are used.

The concentration of purified water or free water in the nutritional composition is relatively low (e.g., compared to standard formulas not designed for use in patients with CKD or AKI) in order to support patients with fluid restrictions.

In general, according to one aspect, the invention features a nutrient composition for nutrition support for patients with chronic kidney disease or acute kidney injury, the nutrient composition being in liquid form for enteral feeding, including tube and/or oral feeding. The nutrient composition comprises pea protein and/or pea protein hydrolysate, phytochemical extracts, fatty acid-containing components, organic ingredients, and prebiotic fibers.

In embodiments, the pea protein and/or pea protein hydrolysate constitutes 8.5 wt % to 12 wt % of the total composition of the total composition and approximately 100 wt % of the total protein of the composition.

The nutrient composition comprises 1000-1400 mg of potassium per liter of the nutrient composition, 1000-1400 mg of sodium per liter of the nutrient composition, 760-800 mg of phosphorous per liter of the nutrient composition, and/or 880-1000 mg of chloride per liter of the nutrient composition.

The nutrient composition also comprises a vitamin and mineral blend, including approximately 8.8-12 mg of vitamin B6 per liter of the nutrient composition, approximately 7.2-10 mg of vitamin B12 per liter of the nutrient composition, approximately 23-28.4 mg of zinc per liter of the nutrient composition, and/or approximately 80-200 mcg of selenium per liter of the nutrient composition.

The prebiotic fibers comprise organic agave inulin constituting approximately 100 wt % of the total dietary fiber of the composition. The organic ingredients include organic agave syrup constituting 2.0 wt % to 5.0 wt % of the total composition, organic brown rice syrup solids constituting 9.0 wt % to 14.0 wt % of the total composition. The fatty-acid containing components comprise organic flax seed oil constituting 0.5 wt % to 2.0 wt % of the total composition, organic high linoleic sunflower oil constituting 2.0 wt % to 4.0 wt % of the total composition, and organic medium chain triglyceride (MCT) oil constituting 2.0 wt % to 4.5 wt % of the total composition.

The nutrient composition further comprises purified water constituting approximately 67 wt % of the total composition. The nutrient composition has a caloric density of 1.8 kcal/mL, a caloric distribution of 18% protein, 38% carbohydrates, and 44% fat, and a glycemic index of 42+/−5.

In general, according to another aspect, the invention features a method of preventing, reducing, and/or treating chronic kidney disease or acute kidney injury. The method comprises administering a nutrient composition to a human subject in need thereof. The nutrient composition, which is in liquid form for enteral feeding, comprises pea protein and/or pea protein hydrolysate, phytochemical extracts, fatty acid-containing components, organic ingredients, and prebiotic fibers.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The disclosed nutritional composition is a low-allergic nutritional composition which is composed of organic and plant-based ingredients. In one embodiment, the nutritional composition comprises at least one protein source, phytochemical extracts, one or more fatty acids, one or more non-allergic organic ingredients, and one or more prebiotic fibers. The organic ingredients are free of corn. The nutritional composition further comprises one or more vitamin and mineral blends, and purified water.

Typically, the nutritional composition further comprises organic brown rice syrup solids and organic agave syrup along with possibly organic vanilla flavor or other flavors.

The nutritional composition is provided in liquid form for enteral feeding, including tube and/or oral feeding, and can be used as a sole source of nutrition or supplemental source of nutrition. For tube feeding, the composition can be administered via pump, gravity, or bolus. In one embodiment, the nutritional composition is provided in cartons, each of which contains approximately 250 mL of the composition.

The present invention also provides an easily digestible organic plant-based peptide, which is a formulation of a vegan product free from the top eight allergens and corn-derived carbohydrates. Specifically, the composition contains no milk, wheat, soybeans, eggs, peanuts, tree nuts, fish and shellfish, is free of corn-derived carbohydrates, and is non-GMO (free of non-genetically modified organisms).

The plant-based protein source is preferably organic pea protein (e.g., from organic yellow pea), including intact pea protein and/or hydrolyzed pea protein. The concentration of the pea protein (e.g., pea protein isolate and/or pea protein hydrolysate) ranges from 7.5 wt % to 13 wt % of the total composition, or preferably 8.5 wt % to 12 wt %. The pea protein constitutes 95% or greater of the total protein of the composition, or preferably 100% of the total protein.

Nevertheless, the composition provides a complete amino acid profile.

Pea protein is high in branched chain amino acids lysine and arginine, which, for example, is helpful in promoting wound healing of diabetic ulcers. Thus, the nutritional composition includes arginine for nitric oxide generation and many other essential benefits including toxin elimination, immune system maintenance, and to support nutritional needs of individuals with wounds for faster wound healing, to name a few examples. The nutritional composition includes 6.4-8.0 g of arginine per 1 L of composition, or preferably approximately 7.2 g/L.

The nutritional composition does not contain any lactose or milk protein.

The composition provides a Protein Digestibility Corrected Amino Acid Score (PDCAAS) ranging from 0.95 to 1.0, or preferably 1.0.

The composition also includes an organic phytonutrient blend, which comprises one or more phytochemical extracts. In one embodiment, the phytochemical extract is a blend of fruits, vegetables, herbs and spices and/or a combination of phytochemical extracts from fruits, vegetables, herbs, and spices. In some embodiments, the blend of phytochemical extracts is sourced from a group of fruits, vegetables, herbs, and spices, comprising one or more concentrates of broccoli sprouts, camu camu, tomato, acai, turmeric, garlic, basil, oregano, cinnamon, elderberry, chokeberry, raspberry, spinach, kale, brussels sprouts, extracts of green arabica coffee bean, green tea, onion, apple, acerola, Japanese pagoda tree (quercetin), blackcurrant, blueberry, bilberry or any combination thereof. In one embodiment, the phytonutrient blend includes extracts and concentrates from: organic broccoli, organic whole fruit of the coffee plant, organic green tea, organic turmeric, organic kale, organic broccoli sprout, organic acai, organic cinnamon, organic garlic, organic tomato, organic blueberry, organic carrot, organic beet, organic raspberry, organic spinach, organic tart cherry, and organic blackberry. The phytonutrient blend can also include green tea extract, green coffee bean extract, maqui berry concentrated juice powder, grape seed extract, carrot juice powder, turmeric powder, cinnamon, broccoli sprout powder, garlic extract, and/or lycopene. The nutritional composition comprises a phytonutrient blend at a concentration ranging from 0.02 wt % to 0.08 wt %, or preferably 0.03 wt % to 0.06 wt %. The nutritional composition comprises a phytonutrient blend at a concentration ranging from 300 mg to 600 mg of phytonutrient blend per 1 L of composition. In the current embodiment, the nutritional composition comprises a phytonutrient blend of 400 mg of phytonutrient blend per 1 L of composition.

In addition, the composition further includes organic rosemary extract at a concentration ranging from 0.02 wt % to 0.04 wt %, or preferably 0.025 wt % to 0.035 wt %. The rosemary-based extract is a natural oxidation-inhibiting product.

In general, these natural ingredients effect biological changes of oxidative and nitrosative stress markers (free radicals), nitric oxide levels, and cellular metabolic activity.

The composition also preferably includes organic MCT oil. MCT is easily digestible, which acts as an immediate energy source via portal vein. The nutritional composition comprises organic medium chain triglycerides (e.g., MCT oil) at a concentration ranging from 1.5 wt % to 5 wt %, or preferably 2.0 wt % to 4.5 wt %. In some embodiments, the MCT is a form of saturated fatty acid from coconut with medium-chain fats between 6-12 carbons.

The composition further includes organic high linoleic sunflower oil and organic flaxseed oil in combination with the MCT oil. This provides a balanced blend of essential omega-6 such as sunflower and flax and omega-3 fatty acids such as ALA (alpha-linolenic acid) from flax, and easily digestible medium chain triglycerides (MCT) from the coconut oil. The omega-6 fatty acid is an essential fatty acid. Omega-3 has many anti-inflammatory properties. The MCT and long chain triglycerides (LCT) ratio ranges from 30:70 and 50:50, and the omega-6 and omega-3 ratio ranges from 3:1 and 4:1. In one embodiment, the MCT and long chain triglycerides (LCT) ratio is optimized to 40:60, and the omega-6 and omega-3 ratio is formulated to 3.5:1.

The composition comprises organic sunflower lecithin at a concentration ranging from 0.1 wt % to 0.7 wt %, or preferably 0.3 wt % to 0.6 wt %. Lecithin is a naturally occurring substance found in the membranes of living cells. Lecithin is extracted from sunflower seeds.

The composition comprises organic flaxseed oil at a concentration ranging from 0.3 wt % to 2.5 wt %, or preferably 0.5 wt % to 2.0 wt %. The organic flaxseed oil is a colorless to yellowish oil obtained from the dried, ripened seeds of the flax plant. The organic flax seed oil is a colorless to yellowish oil obtained from the dried, ripened seeds of the flax plant.

The composition comprises organic high linoleic sunflower oil at a concentration ranging from 1.5 wt % to 5.0 wt %, or preferably 2.0 wt % to 4.0 wt %. The organic high linoleic sunflower oil is a variety of sunflower oil containing nearly 70% polyunsaturated linoleic acid.

The composition also preferably includes organic vanilla plant extract at a concentration ranging from 0.6 wt % to 2 wt %, or preferably 1.0 wt % to 1.5 wt %. The organic vanilla plant extract is derived from orchid seed pods of the genus Vanilla. The vanilla plant extract contains several medicinal properties, which could be useful for tackling several ailments. It is also a rich constituent of flavonoids, alkaloids, glycosides, carbohydrates, and several other phytochemicals. The vanilla plant extract also contains both analgesic, antispasmodic, antioxidant and anti-inflammatory properties.

The nutritional composition does not include any artificial sweeteners, sugar alcohols, artificial flavors, or corn syrup.

The composition also includes a vitamin and mineral blend at a concentration ranging from 0.4 wt % to 1.2 wt %, or preferably 0.5 wt % to 0.8 wt %. In the current embodiment, the blend includes Calcium Carbonate, Potassium Chloride, Choline Bitartrate, Sodium Chloride, Sodium Ascorbate, L-Carnitine, L-Cysteine, Ferric Pyrophosphate, Zinc Oxide, DL-Alpha-Tocopheryl Acetate, Beta Carotene, Calcium Pantothenate, Niacinamide, Sodium Molybdate, Copper Sulfate, Manganese Sulfate, Chromium Picolinate, Sodium Selenite, Taurine, Vitamin A Palmitate, Biotin, Pyridoxine Hydrochloride, Folic Acid, Thiamine Hydrochloride, Riboflavin, Phytonadione, Potassium Iodide, Cyanocobalamin (Vitamin B12), Cholecalciferol (Vitamin D3), and Phylloquinone. In one embodiment, the vitamin and mineral blend comprises at least potassium citrate, magnesium citrate, and/or potassium chloride.

In one embodiment, the nutritional composition provides nutrients as indicated in Table 2 below.

TABLE 2
Typical Nutrient Amounts of Exemplary Nutritional Composition

| | | AMOUNT | AMOUNT |
|---|---|---|---|
| | | PER CARTON | PER LITER |
| NUTRIENT COMPOSITION | | (250 mL) | (1000 mL) |
| CALORIES | kcal | 450 | 1800 |
| TOTAL FAT¹ | g | 22 | 88 |
| SODIUM | mg | 250 | 1000 |
| POTASSIUM | mg | 250 | 1000 |
| TOTAL CARBOHYDRATE | g | 43 | 172 |
| DIETARY FIBER | g | 4 | 16 |
| SUGARS | g | 12 | 48 |
| PROTEIN | g | 20 | 80 |
| VITAMIN A | | | |
| PALMITATE | mcg RAE | 90 | 360 |
| BETA CAROTENE | mcg RAE | 200 | 800 |
| VITAMIN C | mg | 25 | 100 |
| CALCIUM | mg | 300 | 1200 |
| IRON | mg | 6 | 24 |
| VITAMIN D | IU mcg | 300/7.5 | 1200/30 |
| VITAMIN E | IU mg | 8.0/3.6 | 32/14 |
| VITAMIN K | mcg | 25 | 100 |
| THIAMIN | mg | 0.5 | 2.0 |
| RIBOFLAVIN | mg | 0.7 | 2.8 |
| NIACIN | mg | 5 | 20 |
| VITAMIN B6 | mg | 2.2 | 8.8 |
| FOLATE | mcg DFE | 425 | 1700 |
| VITAMINS B12 | mcg | 1.8 | 7.2 |
| BIOTIN | mcg | 15 | 60 |
| PANTOTHENIC ACID | mg | 3.7 | 15 |
| PHOSPHORUS | mg | 190 | 760 |
| IODINE | mcg | 40 | 160 |
| MAGNESIUM | mg | 45 | 180 |
| ZINC | mg | 5.8 | 23 |
| SELENIUM | mcg | 20 | 80 |
| COPPER | mg | 0.5 | 2.0 |
| MANGANESE | mg | 0.5 | 2.0 |
| CHROMIUM | mcg | 30 | 120 |
| MOLYBDENUM | mcg | 20 | 80 |
| CHLORIDE | mg | 220 | 880 |
| CHOLINE | mg | 160 | 640 |
| L-CARNITINE | mg | 65 | 260 |
| TAURINE | mg | 38 | 150 |

¹8.4 g MCT/250 mL; 33.6 g MCT/1000 mL.

The composition further comprises purified water at a concentration ranging from 55 wt % to 75 wt %, or preferably 60 wt % to 70 wt %. In the current embodiment, the concentration of purified water is 64 wt %.

The composition also includes organic locust bean gum at a concentration ranging from 0 wt % to 0.2 wt %, or preferably 0.02 wt % to 0.1 wt %.

The composition also includes prebiotic fibers and particularly organic agave inulin. The organic Agave inulin is a highly soluble dietary fiber (inulin-type fructan) that provides numerous health benefits. Inulin serves as a “fertilizer” to sustain beneficial bifidobacteria in the large intestine.

Prebiotic fiber is clinically proven to be easy to digest (low gas potential) and promote the growth of good bacteria in the gut such as bifidobacteria. Further, Fecal Actinobacteria and bifidobacterium were enriched 3- and 4-fold after ingestion of agave inulin, a long chain fructo-oligosaccharide (FOS), and desulfovibrio were depleted 40% with agave inulin. Agave inulin tended to reduce fecal 4-methyphenol and pH. A positive association between intakes of agave inulin and Bifidobacterium is briefly described herein. Total dietary fiber from agave inulin was positively associated with fecal butyrate and tended to be positively associated with bifidobacterium and was negatively correlated with desulfovibrio abundance. FOS could foster Bifidobacterium colony growth, which could also serve as food for less desirable strains of bacteria. Chicory inulin, a source of prebiotic fiber, encourages the growth of klebsiella, which causes problems with intestinal permeability. It also feeds E. coli and many Clostridium species, which are inharmonious with gut-friendly bacteria.

The nutritional composition further comprises organic agave syrup at a concentration ranging from 1.5 wt % to 6 wt %, or preferably 2.0 wt % to 5.0 wt %. The organic agave syrup constitutes 30% to 70% of the total sugars of the composition, or preferably approximately 52% of the total sugars. The organic agave syrup is a natural alternative to refined sugars and artificial sweeteners, extracted from the agave plant.

The nutritional composition further comprises organic brown rice syrup solids at a concentration ranging from 7 wt % to 16 wt %, or preferably 9.0 wt % to 14.0 wt %. The organic brown rice syrup solids constitute 30% to 70% of the total sugars of the composition, or preferably approximately 46% of the total sugars.

In one embodiment, the total sugars of the composition consist of approximately 48 g of sugars per 1 L of composition.

The composition also includes organic pea starch at a concentration ranging from 7 wt % to 14 wt %, or preferably 9 wt % to 12 wt %.

The organic agave syrup, organic brown rice syrup solids, and organic pea starch constitute 90% to 100% of the total carbohydrates of the composition, or preferably approximately 98% of the total carbohydrates.

The nutritional composition contains a blend of complex and simple carbohydrates to create a more favorable blood glucose response. For example, the organic agave syrup is fructose-based, which helps contribute to a low glycemic index of the overall product, which ranges from 35 to 55. In one embodiment, the glycemic index is 42+/−5.

More particularly, the agave syrup contains a higher level of fructose than high fructose corn syrup. An important difference is that fructose is up to twice as sweet as sucrose, and sweeter than high fructose corn syrup. Unlike table sugar or HFCS, fructose does not cause a rapid rise and subsequent large fall in blood glucose levels, which means it has a low glycemic load or glycemic index (GI). The glycemic index is a value assigned to foods based on how slowly or how quickly those foods cause increases in blood glucose levels. The blood glucose levels above normal are toxic and can cause blindness, kidney failure, or increase cardiovascular risk. Food components that are low on the glycemic index (GI) scale tend to release glucose slowly and steadily. Food components that are high on the glycemic index release glucose rapidly. Low GI foods tend to foster weight loss, while foods high on the GI scale help with energy recovery after exercise, or to offset hypo- (or insufficient) glycemia. For an average diet, there are no health problems associated with fructose except for some individuals, who may be fructose intolerant.

Fructose was given GRAS status in 1983 and was reconfirmed in 1996 after a study of the available clinical trials on the effects of fructose consumption. Fructose has a low glycemic index, which can be used to help control blood glucose levels in concern with a total diet plan. Agave syrup is not processed in the same manner as corn syrup. Agave naturally contains inulin, a long chain fructose molecule. Fructose, sometimes known as fruit sugar, exists naturally in fruits and honey as a single unit of fructose. It exists naturally in inulin as a long chain of fructose molecules linked together, similar to the glucose chains in starch. The bonds in inulin can be broken to make free fructose. When a person eats a food containing inulin, the body's enzymes do not break down the inulin. Instead, the inulin is consumed by bacteria in the intestine. The bacteria that consume the inulin tend to be the beneficial bacteria, the same types found in yogurts and various probiotic foods. As the inulin feeds these beneficial organisms, allowing them to outcompete the detrimental intestinal organisms, inulin is labeled a prebiotic and has many health benefits. While fructose could be made by hydrolyzing or breaking the bonds of inulin, it could also be made by using an isomerase enzyme to change glucose into fructose.

The organic brown rice syrup solid (BRSS) is a natural alternative to refined sugars and artificial sweeteners from the agave plant. Unlike refined table sugar, which causes the body to work harder to absorb the sucrose, brown rice syrup is able to provide body with fiber, as well as 3% of a user's recommended daily intake of sodium and potassium. As the rice syrup solids are boiled and the liquid are removed, the result is a complex of simple sugars. This facilitates slower absorption of the sugars in rice syrup by the digestive system. This is a huge advantage because in the case of regular table sugar, there is rapid swell in blood sugar level with buildup of excess fat with time. The slower absorption of the sugars also allows the user or patient to maintain their energy and prevents the negative effects of sugar including fatigue, irritability, and the desire for more sugar.

These properties indicate that BRSS have a low glycemic index. Use of the natural sweetening ability of BRSS is unique in the space of medical grade sole source nutrition. BRSS is a far superior option than maltodextrin, commonly used as a carbohydrate in comparable products. Maltodextrin is used as a thickener, filler, or preservative in many processed foods. It is an artificially produced white powder that can be enzymatically derived from any starch but is commonly made from corn, rice, potato starch or wheat. Maltodextrin is used in carbohydrate supplements marketed to athletes and bodybuilders to boost their energy levels. However, consuming maltodextrin may lead to spiked blood sugar, suppression of growth of certain probiotics, may have toxic impact on several bodily organs and systems, and cause allergic reactions or side effects in susceptible individuals due to its derivation from corn.

The organic flax seed oil has a high concentration of healthy omega-3 s as fatty acids associated with brain and heart health, mood support, decreased systemic inflammation, and healthier skin and hair. Flax seed oil contains 50% to 60% of omega-3 fatty acids in the form of alpha-linolenic acid (ALA). Flax seed is the richest plant source of the omega(co)-3 fatty acid, i.e. a-linolenic acid (ALA). Flax seed oil is low in saturated fatty acids (9%), moderate in monosaturated fatty acids (18%), and rich in polyunsaturated fatty acid (73%). Of all lipids in flaxseed oil, a-linolenic acid is the major fatty acid ranging from 39% to 60.42% followed by oleic, linoleic, palmitic and stearic acids, which provides an excellent omega (co)-6:omega (co)-3 fatty acid ratio of approximately 0.3:1. Flax seed oil is naturally high in antioxidants like tocopherols and beta-carotene. Flax seed oil provides basic nutrition as well as health benefits for reduction of cancer risk, heart health support and reducing cardiovascular disease risk by lowering plasma LDL-cholesterol and enhancing vasodilatory functions. Therefore, flax seed oil could be considered as a functional food ingredient. Flax seed oil is emerging as an important functional food ingredient, because of its rich contents of a-linolenic acid (ALA, omega-3 fatty acid), lignans, and fiber. Flax seed oil, fibers and flax lignans have potential health benefits such as, in reduction of cardiovascular disease, atherosclerosis, diabetes, cancer, arthritis, osteoporosis, autoimmune and neurological disorders.

The organic high linoleic sunflower oil comprises essential omega-6, impressive fatty acid content, which includes palmitic acid, stearic acid, oleic acid, and linoleic acid. The combination of fatty acids in the body are extremely important to maintain various elements of human health, and sunflower oil could help to maintain the balance. The balance of fatty acids found in sunflower oil, including a substantial amount of linoleic acid (an omega-6 fatty acid) is optimal for patients. Finding a healthy balance between HDL or good cholesterol (omega-3 s) and LDL or bad cholesterol is very important to the health of patients. Furthermore, sunflower oil does not contain any saturated fats, which means that it may reduce overall plasma cholesterol. Omega-6 (n-6) polyunsaturated fatty acids (PUFA) in High Linoleic Sunflower Oil (e.g., arachidonic acid (AA)) and omega-3 (n-3) PUFA (e.g., eicosapentaenoic acid (EPA)) are precursors to potent lipid mediator signaling molecules, termed “eicosanoids,” which have important roles in the regulation of inflammation.

The composition typically is engineered to have an optimal combination of omega-3 and omega-6 fatty acids which are types of essential fatty acids that could not be prepared on our own, which have to obtain them from diet. Both are polyunsaturated fatty acids that differ from each other in their chemical structure. There are two critical omega-3 fatty acids, (EPA and DHA), that the patients require. EPA and DHA are the building blocks for hormones that control immune function, blood clotting, and cell growth as well as components of cell membranes.

In contrast, omega-6 foods containing these fatty acids are numerous in modern diets. They are found in seeds and nuts, and the oils are extracted from them. The body also constructs hormones from omega 6 fatty acids. In general, hormones derived from the two classes of essential fatty acids have opposite effects. Those from omega-6 fatty acids tend to increase inflammation (an important component of the immune response), blood clotting, and cell proliferation, while those from omega-3 fatty acids decrease those functions. Both families of hormones must be in balance to maintain optimum health, so there are some omega-6 benefits. Many nutrition experts believe that before we relied so heavily on processed foods, humans consumed omega-3 and omega-6 fatty acids in roughly equal amounts. The imbalance between omega-3 and omega-6 fatty acids may also contribute to obesity, depression, dyslexia, hyperactivity and even a tendency toward violence. Bringing the fats into proper proportion may actually relieve those conditions. The composition of the present invention has a unique 3.5:1 ratio of omega 6:omega 3 ratio, which facilitates balance of these fatty acids and contributes to the anti-inflammatory effects of them.

The agave inulin is a highly soluble dietary fiber (inulin-type fructan) that provides numerous health benefits. Inulin serves as a “fertilizer” to sustain beneficial bifidobacteria in the large intestine. Inulin is clinically proven to be easy to digest and promotes the growth of good bacteria in the gut such as bifidobacteria. In studies, fecal actinobacteria and bifidobacteria were enriched 3- and 4-fold after ingestion of agave inulin, a long chain fructo-oligosaccharide (FOS), and desulfovibrio were depleted 40% with agave inulin compared with the control. Agave inulin tended to reduce fecal 4-methyphenol and pH. Total dietary fiber from agave inulin has been positively associated with fecal butyrate and tended to be positively associated with Bifidobacterium and was negatively correlated with desulfovibrio abundance.

The MCT (Medium-Chain Triglycerides) oil is quickly and easily digested and provides a relatively large amount of energy to the patient. It is associated with stimulation of the metabolism and has been identified as a potential weight loss aid. If MCT oil is consumed to increase weight gain, a substantial amount of protein would aid in weight gain. Medium-chain triglycerides are easily absorbed by the gastrointestinal tract and converted into energy by the liver. They are often digested well even by those who cannot digest normal, long-chain fatty acids. MCT is comprised of primarily caprylic and capric fatty acids. MCT helps to treat diseases such cystic fibrosis, and fat malabsorption. It has also been used to add calories to infant and certain other formulas. Unlike other fats, MCT oil benefits because it does not go through the lymphatic system, it is transported directly to the liver where it is metabolized so it releases energy like a carbohydrate and creates ketones.

The organic sunflower lecithin is good for heart health. It helps to prevent and reverse damage from coronary disease. Lecithin is essentially an emulsifier of fat which could be used by the body to discard the excess fat from the bloodstream. These excess fats include cholesterol and triglycerides meaning that it will help you to reduce and control the levels of cholesterol. High concentrations of linoleic acid present in the sunflower lecithin help perform the above process. In addition, olive oil has similar levels of linoleic acid. Phospholipids also plays a vital essential role for liver health and prevents excessive accumulation of fat.

Another advantage of using sunflower lecithin is the extraction process. Sunflower lecithin gets extracted without the need for potentially damaging chemical solvents such as acetone and hexane. Sunflower lecithin is produced through a cold press system, as like deriving oil from olives and similar products. Sunflower lecithin is abundant with essential choline and fatty acids, such as phosphatidylinositol.

The organic vanilla plant extract soothes inflammation throughout the body. This is particularly effective for inflammation of the liver, which occurs due to numerous reasons, especially for immunocompromised individuals. Vanillin could help ease arthritis, gout and other inflammatory conditions. Vanilla extract has been evaluated for hepatoprotective activity against paracetamol-induced liver damage in rats. The researchers orally administered an ethanolic extract of Vanilla planifolia or a control reference solution to experimental animals with hepatotoxicity induced by paracetamol. It was observed that the plant extract was able to protect the liver against the injury induced by paracetamol in rats. This was proven by the tremendous reduction in serum enzymes alanine aminotransferase (ALT), aspartate aminotransferase (AST), alkaline phosphatase (ALP) and bilirubin. The ethanolic extract of Vanilla planifolia has hepatoprotective activity against paracetamol-induced hepatotoxicity in rats. Vanilla extract components were studied for the potential use as antioxidants for food preservation and as nutraceuticals in health supplements. The extract and pure standard compounds were screened to ascertain their antioxidant properties using b-carotene-linoleate and diphenyl picryl hydrazyl (DPPH) in vitro model systems. Interestingly, the study observed and supported the potential use of vanilla bean extracts as antioxidants.

Phytochemicals are the active ingredients that promote whole body health. The extracts do not contain any source ingredients such as, protein, fat, or carbohydrates. In one embodiment, the blend may include extracts of green tea and green coffee but without containing any caffeine. Blend of phytochemical extracts from fruits, vegetables, herbs, and spices represents the latest evolution in the fight against potentially damaging free radicals. The biological effects of a natural supplement on the changes of oxidative and nitrosative stress markers, as well as cellular metabolic activity, have been clinically observed in the human body. The blend of phytochemical extract has been reported to decrease ROS, increase cellular oxygen consumption in blood and mitochondria, decrease extracellular H₂O₂, and reduce TNFa-induced inflammatory response in humans. Administration of blend of phytochemical extract resulted in statistically significant long-term inhibition of mitochondrial and cellular ROS generation by as much as 17% as well as 3.5-times inhibition in extracellular NADPH system-dependent generation of O₂, and nearly complete inhibition of extracellular H₂O₂ formation. This was reflected in more than two times inhibition of ex vivo cellular inflammatory response and increases in bioavailable NO concentration. Further, there was measured synergetic, biological effects of a natural supplement on changes in OSM and cellular metabolic activity. The unique design and activity of the plant-based natural supplement, in combination with the newly developed and extended Vitality Test, demonstrates the potential of using dietary supplements to modulate OSM and also opens the door to future research into the use of natural supplements for supporting optimal health.

Potassium chloride provides benefits and relief related to strokes, high blood pressure, heart and kidney disorders, and anxiety and stress. It helps in enhancing muscle strength, metabolism, water balance, electrolytic functions, and nervous system. Chloride works with other electrolytes, such as sodium and potassium, to help balance acids and bases in the body. It also helps to move fluid in and out of the cells, which is also essential for preventing dehydration.

Advantageously, the nutritional composition is entirely plant-based and free of the top eight allergens such as milk, wheat, soybeans, eggs, peanuts, tree nuts, fish and shellfish, corn, and is non-GMO (free of non-genetically modified organisms). In addition, it contains organic ingredients, optimal fatty acid ratios, oxidation fighting ingredients, anti-inflammatory properties, and beneficial fiber. Alternative approaches to the problem include formation of a blended food product.

In the current embodiment, the nutritional composition has: 1800 kcal per 1 L of the composition with a caloric density of 1.8 kcal/mL or 1800 kcal/L; a caloric distribution of 18% protein, 38% carbohydrates, and 44% fat; 80 g of protein per 1 L of the composition from a plant-based protein source, namely organic pea protein; 170-172 g of carbohydrates per 1 L of the composition from organic brown rice syrup solids, organic agave syrup, and organic pea starch; 88 g of fat per 1 L of the composition from organic MCT oil, organic high linoleic sunflower oil and organic flaxseed oil, 16 g of dietary fiber per 1 L of the composition from organic agave inulin; 48 g of sugar per 1 L of the composition from organic brown rice syrup solids, and organic agave syrup; an MCT:LCT ratio of 40:60; an osmolality (mOsm/kg water) of 360-370; a renal solute load of 558 mOsm/L; an omega 6:3 ratio of 3.5:1; a free water percentage of 67%; 400-600 mg of phytonutrients per 1 L of the composition; a nonprotein calorie to nitrogen ratio of 116:1; a glycemic index of 42+/−5; 1000 mg of sodium per 1 L of the composition; 1000 mg of potassium per 1 L of the composition; 760 mg of phosphorous per 1 L of the composition; and 880-900 mg of chloride per 1 L of the composition.

While this invention has been particularly shown and described with references to preferred embodiments thereof, it will be understood by those skilled in the art that various changes in form and details may be made therein without departing from the scope of the invention encompassed by the appended claims.

What is claimed is:

1. A nutrient composition for nutrition support for patients with chronic kidney disease or acute kidney injury, the nutrient composition being in liquid form for enteral feeding and comprising:
pea protein and/or pea protein hydrolysate;
phytochemical extracts;
fatty acid-containing components;
organic ingredients; and
prebiotic fibers.

2. The nutrient composition of claim 1, wherein the pea protein and/or pea protein hydrolysate constitutes 8.5 wt % to 12 wt % of the total composition.

3. The nutrient composition of claim 2, wherein the pea protein and/or pea protein hydrolysate constitutes approximately 100 wt % of the total protein of the composition.

4. The nutrient composition of claim 1, wherein the nutrient composition has a caloric density of 1.8 kcal/mL.

5. The nutrient composition of claim 1, wherein the nutrient composition comprises 1000 mg to 1400 mg of potassium per liter of the nutrient composition.

6. The nutrient composition of claim 1, wherein the nutrient composition comprises 1000 mg to 1400 mg of sodium per liter of the nutrient composition.

7. The nutrient composition of claim 1, wherein the nutrient composition comprises 760 mg to 800 mg of phosphorous per liter of the nutrient composition.

8. The nutrient composition of claim 1, wherein the nutrient composition comprises 880 mg to 1000 mg of chloride per liter of the nutrient composition.

9. The nutrient composition of claim 1, further comprising a vitamin and mineral blend.

10. The nutrient composition of claim 9, wherein the vitamin and mineral blend comprises approximately 8.8 mg to 12 mg of vitamin B6 per liter of the nutrient composition.

11. The nutrient composition of claim 9, wherein the vitamin and mineral blend comprises approximately 7.2 mg to 10 mg of vitamin B12 per liter of the nutrient composition.

12. The nutrient composition of claim 9, wherein the vitamin and mineral blend comprises approximately 23 mg to 28.4 mg of zinc per liter of the nutrient composition.

13. The nutrient composition of claim 9, wherein the vitamin and mineral blend comprises approximately 80 mcg to 200 mcg of selenium per liter of the nutrient composition.

14. The nutrient composition of claim 1, wherein the prebiotic fibers comprise organic agave inulin constituting approximately 100 wt % of the total dietary fiber of the composition.

15. The nutrient composition of claim 1, wherein the organic ingredients include organic agave syrup constituting 2.0 wt % to 5.0 wt % of the total composition.

16. The nutrient composition of claim 1, wherein the organic ingredients include organic brown rice syrup solids constituting 9.0 wt % to 14.0 wt % of the total composition.

17. The nutrient composition of claim 1, wherein the fatty-acid containing components comprise organic flax seed oil constituting 0.5 wt % to 2.0 wt % of the total composition.

18. The nutrient composition of claim 1, wherein the fatty-acid containing components comprise organic high linoleic sunflower oil constituting 2.0 wt % to 4.0 wt % of the total composition.

19. The nutrient composition of claim 1, wherein the fatty-acid containing components comprise organic medium chain triglyceride (MCT) oil constituting 2.0 wt % to 4.5 wt % of the total composition.

20. The nutrient composition of claim 1, further comprising purified water constituting approximately 67 wt % of the total composition.

21. The nutrient composition of claim 1, wherein the nutrient composition has a caloric distribution of 18% protein, 38% carbohydrates, and 44% fat.

22. The nutrient composition of claim 1, wherein the nutrient composition has a glycemic index of 42+/−5.

23. A method of preventing, reducing, and/or treating chronic kidney disease or acute kidney injury, the method comprising administering to a human subject in need thereof a nutrient composition, the nutrient composition being in liquid form for enteral feeding and comprising:
pea protein and/or pea protein hydrolysate;
phytochemical extracts;
fatty acid-containing components;
organic ingredients; and
prebiotic fibers.
